(19) United States
(12) Plant Patent Application Publication
(10) Pub. No.: US 2005/0039236 P1
(43) Pub. Date: Feb. 17, 2005

(54) Grape plant named 'G1-6819'

(50) VITIS SP.; GRAPE

(76) Inventors: Philipus J.L. Ellis, Stellenbosch (ZA); Phyllis Burger, Stellenbosch (ZA)

Correspondence Address: CHRISTIE, PARKER & HALE, LLP, PO BOX 7068, PASADENA, CA, 91109-7068, US

(21) Appl. No.: 10/857,045

(22) Filed: May 28, 2004

(30) Foreign Application Priority Data
Jun. 1, 2003 (ZA) PT 3838

(51) Int. Cl. A01H 5/00

(52) U.S. Cl. PLT/205

(57) ABSTRACT

A new and distinct variety of Grapevine, designated ‘G1-6819’, characterized by strong vegetation, on own root and when grafted, high yield and large bunches, fire bright red berry color with very uniform coverage and color that does not get darker as ripening goes on, somewhat tight bunches, and very uniform berries, meaty with no water berries, that do not stutter or crack.

### BOTANICAL CLASSIFICATION/CULTIVAR DENOMINATION

Vitis sp. cultivar ‘G1-6819’.

### BACKGROUND OF THE INVENTION

The present invention relates to a new and distinct variety of Grapevine, botanically known as Vitis sp., and hereinafter referred to by the name ‘G1-6819’.

‘G1-6819’ was bred by cross pollination. It has been asexually reproduced by tissue culture in South Africa. Observations made on ‘G1-6819’ since September 2003 have shown that the unique features of this new Grapevine are stable and reproduced true to type in successive generations.

### SUMMARY OF THE INVENTION

The following traits have been repeatedly observed and are determined to be the unique characteristics of ‘G1-6819’. All observations were made from specimens grown in Kern County, Calif., during September 2003. These characteristics in combination distinguish ‘G1-6819’ as a new and distinct Grapevine cultivar:
1. Very late ripening variety, typically ripening in September;
2. Medium growth variety, with fairly good production;
3. Nice attractive elongate berry shape, natural loose bunch and large berry size;
4. Holds well in cold storage and transportation; good resistance to rotting

### BRIEF DESCRIPTION OF ILLUSTRATIONS

The accompanying photographic illustrations show typical specimens of the new variety, in color as nearly true as it is reasonably possible to make in a color illustration of this character. Colors in the photograph may differ from the color values cited in the detailed botanical description below, which accurately describes the colors of the new Grapevine.

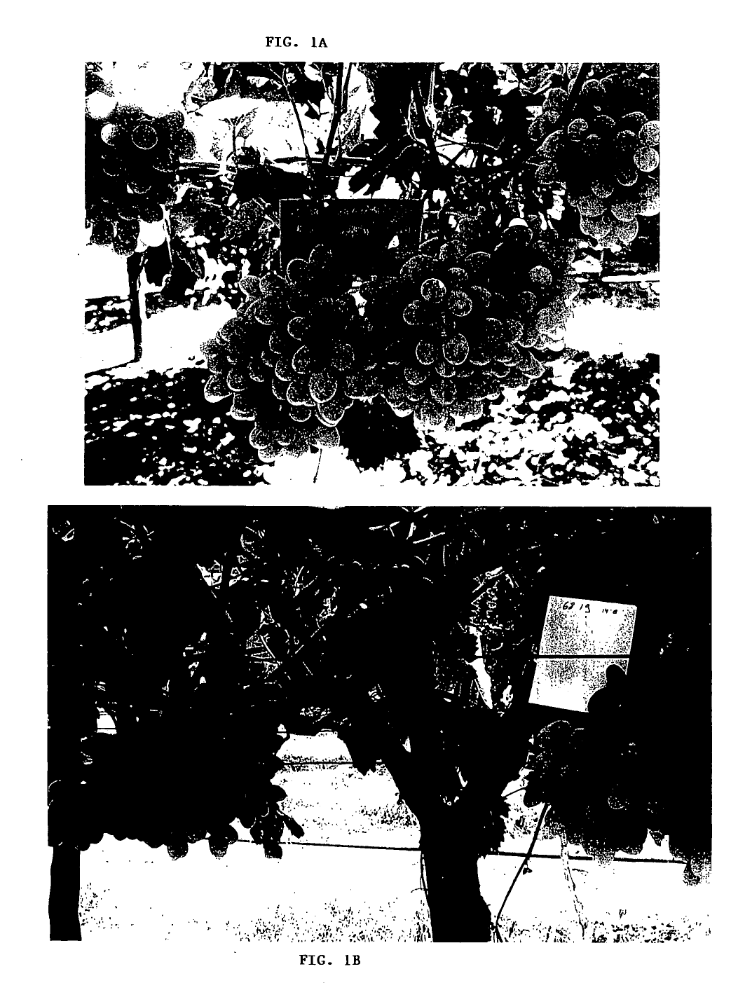

FIG. 1A comprises a close-up of bunches of ‘G1-6819’.

FIG. 1B comprises a close-up of the new grapevine.

### DESCRIPTION OF THE NEW VARIETY

Referring now specifically to the new and distinct variety of Grapevine, the following is a detailed description of ‘G1-6819’ with color description where indicated by reference to The Royal Horticultural Society Colour Chart, except where common terms of color definition are employed.

The new variety of Grapevine as herein described may vary in slight detail due to climatic, soil and cultural conditions under which the variety may be grown, the present description being of the variety as grown at Kern County, Calif., between April and September of 2003.

### PLANT CHARACTERISTIC

Form.—Upright with a moderate canopy.
Growth.—Moderate, vigorous growth, with medium canes and medium trunk.

### VEGETATIVE CHARACTERISTICS

Vine:
   Size.—Medium canopy with medium leaf size.
   Vigor.—Moderate.
   Pruning.—Spur pruning is sufficient due to the good fertility.
   Trunk.—Normal.
   Bark.—Normal smooth, grey-orange color near 165A.
Canes:
   Form.—Smooth, somewhat sparser than UPOV 6.1.41/1.
   Length.—From spur — average mature cane 9′-12′.
   Size.—Variety canes are fully mature at harvest time, up to 80% along the cane the brownish color showing full maturity.
   Color.—Mature cane near 165B, grey orange group, somewhat shiny with red/burgundy dots near 183C grey-purple group.
   Width at center node.—Cane with bunch — ½ inch; Cane without bunch — ⅝ inch.
   Nodes (generally).—Standard node, round smooth and standard look with no deformation. No prostrate hairs and no hairs.
   Width at center internodes.—1 cane with bunch — ⅜ inch; 2. cane without bunch — 7/16 inch.
   Internodes length.—Cane with bunch — 4½ inches; Cane without bunch — 5¼ inches.
Tendrils: In the early stage, the spring tendrils are large and upright, at maturity the tendril break off at the splitting point; very few tendrils remain on the mature cane, three or more.
   Length.—Categorize as short — 3½ inch to the break split point and 3½ inches of curlicue, total of 7 inches for mature leaves, for green and young tendril, third from the top the length is 2½ inches to the break split point and 2½ inches of curlicue.
   Average thickness.—⅜ inches.
   Location.—Mainly from the center to the top of the shoot.
   Leafing.—No leaves grow on the tendrils.
   Color.—Mature tendrils color is grey-orange near 166C; green tendril color is yellow-green near 145A.
   Form.—Splitting into two laterals equally.
   Texture.—Straight and firm up to splitting point, splits to two equal laterals.
Foliage:
   Density (average number of leaves about each cluster).—Up to 28 leaves. The foliate of this particular variety unlike other common varieties is light due to large distance between nodes and medium leaf size.
   Color of immature leaf.—Upper side — green-yellow green near 146C.
   Color of mature leaf.—Upper side is green group near 139A; bottom side is green group color 137C.
   Color of petioles.—Light green on bright yellow background at the bottom of the petiole has small strips and/or purple dots. The petiole main color is green group color near 144A and reddish strips/or dots of red purple group color near 61B.
   Color of veins.—Bottom — yellow-green group near 145B; upper — yellow-green group near 145A.
   Texture of leaf surface.—The leaf texture is smooth and shiny on the immature leaves; the leaf texture is smooth and no hairs on the mature leaves.
   Inferior.—Smooth.
   Superior.—Very smooth surface.
   Average length of immature leaf.—4 inches.
   Average length of mature leaf.—5½ inches.
   Average width of immature leaf.—3¾ inches.
   Average width of mature leaf.—8 inches.
   Overall “shape” of leaf.—Serrated and rounded.
   Description of the leaf edge.—Serrated margin with two indentations approximately ⅜ inch to ½ inch in depth.
Blooming/flowers: Stamens are taller than ovaries which make the flower self fertile.
   Date of first bloom.—Around April 29 in the hot valleys of the northern hemisphere.
   Date of last bloom.—Around May 11 in the south end of the San Joaquin Valley.
   Average size of flowers.—Normal.
   Color and size of pedicel.—Light yellow/green pedicel average length is ⅛ inch.
   Color and size of stamen.—Yellow cream — Naples Yellow; Stamen average length is about ⅛ inch.
   Texture of pedicel.—The flower is butterfly shaped with creamy color.
Fruit cluster: The bunch in general has conical shape, bunch is large, about 2 pounds, in general the bunch is semi loose.
   Ripening period.—Bunch length average is 10-12 inches and average width is 6-7¾ inches, with thick strong main stem with no abscission. Point stem color at maturity on mature canes is yellow-green group near 146C.
   Point stem color at maturity on mature canes.—Yellow-green group near 146C.
   Peduncle color at maturity.—Yellow-green group near 149D, 1 inch long and 3/16 lines — ¼0 inch thick.
   Berry size.—Very uniform in the bunch and from bunch to bunch.
Harvest time/maturity: First week of September through mid September.
   Average number of clusters per cane.—1.5.
   Average bunch weight.—1.7 pounds.
   General description of the berry.—At maturity natural berry size without Gibberelic acid treatment is medium size. The berries are well attached to the cap. The variety has very little shatter.
   Average size of the berry.—The size of non treated berry with Gibberelic acid — Berry size length: 1.0 inch; berry size width: ⅝×1 inch.
   Average berry weight of untreated vine without Gibberelic acid.—4.5 gr.
   Sugar/acid/ratio.—Natural berry with no Gibb — 17*-18* Brix.
   Set of the berry.—Average natural shatter.
   Skin thickness.—Skin thickness — fruit thickness slightly thicker than Thompson seedless.
   Color of skin.—Creamy green color — yellow-green group near 146C.
   Color of pulp.—Clear white crystal clear.
   Texture of berry pulp.—Meaty from one side and juicy from other side.
   Presence of needs.—No seed trace.

1. A new and distinct variety of Vitis sp., identified as ‘G1-6819’, substantially as shown and described.
